(19) United States
(12) Patent Application Publication
(10) Pub. No.: US 2018/0322798 A1
(43) Pub. Date: Nov. 8, 2018

(54) SYSTEMS AND METHODS FOR REAL TIME ASSESSMENT OF LEVELS OF LEARNING AND ADAPTIVE INSTRUCTION DELIVERY

(71) Applicant: Florida Atlantic University Board of Trustees, Boca Raton, FL (US)

(72) Inventors: Hari Kalva, Delray Beach, FL (US); Saurin Parikh, Boca Raton, FL (US)

(21) Appl. No.: 15/968,052

(22) Filed: May 1, 2018

Related U.S. Application Data
(60) Provisional application No. 62/500,753, filed on May 3, 2017.

(51) Int. Cl. G09B 5/12; G06N 99/00; G09B 7/07; G09B 5/06; G09B 17/00; A61B 3/113; A61B 5/0476; A61B 5/00; A61B 5/16; A61B 5/04; A61B 3/11

(52) U.S. Cl. CPC G09B 5/12; G06N 99/005; G09B 7/07; G09B 5/065; G09B 17/003; A61B 3/113; A61B 5/0022; A61B 5/0077; A61B 5/7405; A61B 5/7267; A61B 5/163; A61B 5/04012; A61B 3/112; A61B 5/0476

(57) ABSTRACT

Systems and methods for predicting a user's learning level or an Area Of Concern (“AOC”). The methods comprise: presenting multimedia content to a user of a computing device; collecting, by at least one learning level indicator device, observed sense data specifying the user's behavior while the user views the multimedia content; analyzing the observed sense data to determine a plurality of metric values for each of a plurality of word categories, a plurality of graphical element categories and/or a plurality of concept categories; and using the metric values for predicting the learning level or AOC based on results of the comparing.

### CROSS-REFERENCE TO RELATED APPLICATIONS

This application claims priority to U.S. Application Ser. No. 62/500,753 which was filed on May 3, 2017. The contents of which are incorporated herein by reference in its entirety.

### BACKGROUND

### Statement of the Technical Field

The present disclosure relates generally to computing systems. More particularly, the present disclosure relates to implementing systems and methods for real time assessment of levels of learning and adaptive instruction delivery.

### Description of the Related Art

E-Learning is emerging as a convenient and effective tool for delivering education courses. E-learning classrooms comprise diverse groups of students from various demographics having varying cognitive abilities. The biggest challenge with this model is the lack of effective tools to assess levels of learning. This limitation may cause a difficulty in retaining students. Table-I depicts the statistical data of some of the e-learning service providers and their students retention statistics.

TABLE 1
Indicates the Mass open online courses (MOOC) Drop out %

| | | | | No. of | % of |
|---|---|---|---|---|---|
| | | | | countries | Students |
| MOOC | No. of | | No. of | represented | drop out |
| (eLearning) | Institutional | No. of | Students | by | from the |
| Service Provider | Partners | Courses | (in million) | Students | Courses |
| Coursera.org* [1] | 107+ | 532+ | 5+ | 190+ | 85%-95% |
| Edx.org# [1a] | 60+ | 300+ | 3+ | 226+ | [1b] |

‘*’ and ‘#’ indicates that the data provided is for year 2013 and 2014 respectively.

E-learning course content is normally multimedia content consisting of Text, Videos, Images, and Animation. The cognition and comprehension of such content depends on learner's various skills (such as Mathematical, Logical reasoning, Quantitative analysis and, Verbal skills). These skills vary greatly among learners and is highly dependent on the following factors: demographics; culture; experience; education and biological factors (e.g., cognitive, psychomotor skills, oculomotor dysfunctions, and reading disorders). All these factors together contribute in demonstrating varying levels of learning.

As noted above, many factors contribute to varying levels of learning. The following paragraphs discuss various scenarios in which learning concerns exist.

Scenario A: Normally nonnative English speaking students (while reading) find it difficult to comprehend the meaning of novel or low frequency English words. This is caused due to their inherent weak verbal and cognitive skills. Due to this reason, text comprehension may be greatly impaired.

Scenario B: Native English speaking students having neurobiological, oculomotor dysfunctions or reading disorders are more prone to delayed word or text comprehension.

In both above mentioned Scenarios A and B, in order to understand the given term/concept, learners' may look up the meaning of the novel or low frequency word (Term) from various online dictionaries and retrieve relevant information from other sources in order to understand the term/concept.

Scenario C: Students having weak cognition mostly experience impaired visual perception, poor visual attention to detail, poor visual memory, difficulty in scanning and searching objects in competing backgrounds, and spatial disorientation. All these impairments cause difficulty in comprehending the meaning of textual and/or non-textual term/concept from the given multimedia content.

In above mentioned Scenarios A-C, predicting a difficult term/concept (prediction of Area of Interest (“AOI”), Area of Concern (“AOC”) or Region of Interest (“ROI”)) in real time may enable e-learning systems to determine the level of learning for a given individual or for a group of persons. Based on the predicted level of learning, the learner may be provided with Assistive and Adaptive Learning (“AAL”) content. Biometric signals acquired by Human Computer Interaction (“HCI”) devices have been used to predict levels of learning.

Prior art work has focused mainly on analyzing various eye movement signals in order to assess the learner's cognitive response. Conventional systems employ models that uses eye tracking data to assess the user's Meta cognitive behavior exhibited during interaction with an exploratory learning environment. Free exploration and self-explanation are considered as two learning skills to assess the user's Meta cognitive behavior. The correlation between pupil dilation & cognitive effort is also considered in context of learning. Eye tracking data has also been used for adaptive e-learning in conventional systems.

One conventional solution mainly focuses on adapting to users preferences, knowledge level and does real time tracking of user behavior. The main focus of the conventional framework is to observe the users learning behavior by monitoring their eye response signals such as fixations and saccades. An eLearning environment is created based on eye tracking. Readers' attention within predefined Region of Interest (“ROI”) is monitored. The readers' fixations, blinks and pupil diameter is analyzed in order to predict the cognitive load experienced by the reader within the respective ROI. The conventional system also tracks the tiredness behavior of the user in order to predict the reading disorientation of the reader in specific ROI.

Another conventional solution comprises an eLearning platform. The main focus of the eLearning platform is to analyze the learners' ocular data (such as gaze coordinates, Fixation Durations (“FDs”), and pupil diameters) which is acquired in real time by using eye trackers for the purpose of detecting an emotional and cognitive state of the user. The eLearning platform was specific to mathematical learning.

Another conventional solution is known as iDict. iDict is a language translation system. The iDict system translates content for the user in several languages based on e5learning (enhanced exploitation of eyes for effective eLearning). e5learning has an emotion recognition capability and can detect a high workload, non-understanding situations, and tiredness situations. Other eLearning platforms analyze pupillary response during searching and viewing phases of e-learning activities or use a correlation established between cognition and gaze patterns in order to classify a student as an imager or a verbalizer.

Eye tracking is used to improve e-learning persuasiveness. A functional triad is used to highlight how eye tracking can increase the persuasive power of a technology such as e-learning. In order to estimate the cognitive load and detect understanding problems, the following factors are considered indicators: a number of blinks; a number of fixations; and an arithmetic mean of pupil diameters. The decrease in blinks plus increase in fixations and pupil diameter, indicates high workload or non-understanding phase.

An empathic tutoring software agent has been used to monitor a user's emotions and interest during learning. Feedback is provided about these monitored parameters. The software agent also provides guidance for learning content, based on learners' eye movement data and past experiences. The empathic tutoring agent software mainly analyzes learners' eye gaze data and monitors their emotions and interests. The term “gaze”, as used herein, means to look steadily, intently and with a fixed attention at a specific point of interest.

Multiuser gaze data may be indicative of various reading disorders and various levels of learning which can be used to classify learners into various learning groups. However several ambiguities have been reported for the interpretation of multiuser gaze data. A framework was created to reduce these ambiguities in interpretation of multiuser gaze data. The framework focuses on the two most common gaze data visualization methods (namely, heat maps and gaze plots), and reduces the ambiguity by interpreting multiuser gaze data.

The above described conventional systems detect the learners' learning difficulty as well as the emotional and cognitive state of a learner. The main focus of these conventional systems is tracking the learning experience and predicting an AOI in real time. The learners' ocular data (such as fixations, saccades, blinks and gaze maps) are mainly used as learning difficulty indicators. The term “learning difficulty indicator”, as used herein, refers to psychophysical data inputs of a person collected via HCI devices (e.g., eye trackers, real sense cameras, Electroencephalograms (“EEGs”), and sensor based wearable device). Involuntary indicators of cognitive load (such as heart rate variability, galvanic skin response, facial expression, pupillary responses, and voice behavior and keyboard interaction) have also been assessed in context of learning.

In one conventional system, pupillary response was considered as the main measure of cognitive load. The system was used with an objective to measure cognitive load of a user in real time by using low cost pupilware. The main limitation of pupilware is its failure to detect dark color pupils. Pupillary response was also considered for predicting the effort spent by individual in processing the user interface.

### SUMMARY

The present disclosure generally concerns implementing systems and methods for predicting a user's learning level or an Area Of Concern (“AOC”). The methods comprise: presenting multimedia content to a user of a computing device; collecting, by at least one learning level indicator device, observed sense data specifying the user's behavior while the user views the multimedia content; analyzing the observed sense data to determine a plurality of metric values for each of a plurality of word categories, a plurality of graphical element categories and/or a plurality of concept categories; and using the metric values for predicting the learning level or AOC based on results of the comparing.

In some scenarios, the metric values are used in a previously trained machine learning model for predicting the learning level or AOC. The machine learning model is trained with (A) observed sense data collected while a user is presented with training multimedia content, and/or (B) observed sense data collected from a plurality of users while each user is presented with training multimedia content. The training multimedia content comprises content of different difficulty levels ranging from (i) text content having only common and high frequency words, (ii) text content having combination of high and low frequency words, (iii) text content having high, low frequency and novel words, and (iv) multi-media content along with textual content.

In those or other scenarios, the learning level indicator device includes, but is not limited to, an eye tracker, an Electroencephalogram, a biometric sensor, a camera, and/or a speaker. In the eye tracker cases, the metric values include, but are not limited to, a single fixation duration value, a first fixation duration value, a gaze duration value, a mean fixation duration value, a fixation count value, a spillover value, a mean Saccade Length (“SL”) value, a preview benefit value, a perceptual span value, a mean pupil diameter value of the left eye recorded during a first pass of the text/concept, a mean pupil diameter value of the right eye recorded during the first pass of the text/concept, a regression count value, a second pass time value, a determinism observed value, a lookback fine detail observed value, a lookback re-glance observed value, a mean pupil diameter value of the left eye recorded during reanalysis, and/or a mean pupil diameter value of the right eye recorded during reanalysis.

The word categories comprise a big-size/high-frequency word category, a big-size/low-frequency word category, a big-size/common-word category, a big-size/novel-word category, a mid-size/high-frequency word category, a mid-size/low-frequency word category, a mid-size/common-word category, a mid-size/novel-word category, a small-size/high-frequency word category, a small-size/low-frequency word category, a small-size/common-word category, and/or a small-size/novel-word category. The concept categories comprises a high familiar category, a novel category, and a low familiar category.

In those or other scenarios, the methods also comprises: dynamically selecting supplementary learning content for the user based on the predicted learning level or AOC; and presenting the supplementary learning content to the user via the computing device. Additionally or alternatively, the methods comprise generating a report of the user's learning state or progress based on the predicted learning level or AOC.

The present document also concerns implementing systems and methods for adapting content. The methods comprise: presenting multimedia content to a user of a computing device; predicting, determining and calculating at least one of a level of learning and an area of concern; and modifying the presented multimedia content based on at least one of the level of learning and the area of concern. The multimedia content is modified by: providing a supplementary content that clarifies the multimedia content; and/or providing definitions of one or more terms in the multimedia content.

The present document also concerns implementing systems and methods for grouping learners. The methods comprise: presenting multimedia content to a user of a computing device; predicting, determining and calculating at least one of a level of learning and an area of concern; and creating a group of learners with at least one of a similar level of learning and a similar area of concern. The learners are grouped and placed in a common chat room and/or a common online study space.

### BRIEF DESCRIPTION OF THE DRAWINGS

The present solution will be described with reference to the following drawing figures, in which like numerals represent like items throughout the figures.

FIG. 1 is an illustration of an illustrative architecture for an illustrative system.

FIG. 2 is an illustration of an illustrative architecture for an illustrative computing device.

FIG. 3 is an illustration of an illustrative machine learning model.

FIG. 4 is an illustration of an illustrative term/concept-response map.

FIG. 5 provides an illustration of an illustrative comparison result table.

FIG. 6 is a flow diagram of an illustrative method for predicting a person's learning level and/or reading disorder.

FIG. 7 is an illustration of an illustrative electronic test survey.

FIG. 8 is an illustration of an illustrative displayed visual content.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present solution may be embodied in other specific forms without departing from its spirit or essential characteristics. The described embodiments are to be considered in all respects only as illustrative and not restrictive. The scope of the present solution is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present solution should be or are in any single embodiment of the present solution. Rather, language referring to the features and advantages is understood to mean that a specific feature, advantage, or characteristic described in connection with an embodiment is included in at least one embodiment of the present solution. Thus, discussions of the features and advantages, and similar language, throughout the specification may, but do not necessarily, refer to the same embodiment.

Furthermore, the described features, advantages and characteristics of the present solution may be combined in any suitable manner in one or more embodiments. One skilled in the relevant art will recognize, in light of the description herein, that the present solution can be practiced without one or more of the specific features or advantages of a particular embodiment. In other instances, additional features and advantages may be recognized in certain embodiments that may not be present in all embodiments of the present solution.

Reference throughout this specification to “one embodiment”, “an embodiment”, or similar language means that a particular feature, structure, or characteristic described in connection with the indicated embodiment is included in at least one embodiment of the present solution. Thus, the phrases “in one embodiment”, “in an embodiment”, and similar language throughout this specification may, but do not necessarily, all refer to the same embodiment.

As used in this document, the singular form “a”, “an”, and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

As discussed in the Background section, the conventional solutions use eye movement signals in order to predict a learning difficulty. The accuracy of these conventional solutions is not satisfactory partly because psycholinguistics theory was not considered. For example, psycholinguistics concepts (e.g., lexical processing of words and syntactic parsing of sentences) are not examined by these conventional systems. The conventional solutions also do not examine the effects of reading disorders and oculomotor dysfunctions on cognition.

Instead of just relying on fixed range of fixations (or any other indicators) as is done in the conventional solutions, the present solution provides a more analytical approach for predicting various levels of learning. The analytical approach involves analyzing anticipatory reading behavior, recurrence quantification analysis of scene and reading perception, effects of subjective familiarity and word frequency on cognition, and/or effects of contextual information in deriving meaning of novel or low familiar words. These concepts are examined in order to increase the prediction accuracy of learning concern for learners having reading impairments and learners having no reading impairments.

The present solution solves the problem of assessing levels of learning in real-time with a higher level of accuracy as compared to conventional solutions. The present solution uses devices such as eye trackers for the learning assessment. Eye trackers provide real-time data on a user's response to visual information presented thereto (e.g., via a display screen). The user's response includes eye response patterns and pupil responses. The user's response is associated with words, phrases and/or concepts. The association is defined in a term/concept-response map. Variations in the term/concept-response map over time provides an indication of whether a student has any difficulties in learning. Other biometric devices can also be used for generating term/concept-response maps and making learning assessments. The present solution has broad application in learning. The present solution can also have a direct impact on learning and teaching Exception Student Education (“ESE”) programs in public schools.

A key aspect of the present solution is the temporal analysis of eye response and stimulus (term/concept) that is causing the response. This is based on the hypothesis that variations in eye response to the same concept over time are indicative of levels of learning. Besides eye responses, other biological factors are considered as learning level indicators. These other biological factors include, but are not limited to, neural signals, pupillary responses, and/or facial expressions. An analysis of these learning level indicators may result in a greater prediction accuracy. HCI based devices (e.g., eye trackers, EEGs, real sense cameras, and wearable sensor based devices) are used to acquire data or information for the biological factors.

In some scenarios, the present solution has a multilayered architecture comprising a base layer and several service layers. The base layer's function is to predict an AOI. The service layer functions include: predicting levels of learning; classifying learners into learner groups; providing Assistive and Adaptive Supplementary Learning Content (“A2SLC”); displaying A2SLC content to the learners; and performing any needed language translation.

The following discussions are provided to assist a reader in understanding the theory behind the present solution, as well as certain important aspects thereof.

Correlation Between Reading Behavior, Linguistics Theory And Human Visual System

While reading online content, eyes tend to fixate on specific words and the fixation duration may vary depending on learner's perception of the term/concept. The learner's perception depends upon his(her) cognitive ability, knowledge, past experiences, language skills, and reading skills.

With reference to reading text, the following questions arise.
How do we read content?
Where do we fixate?
Why do we fixate on a word or specific object?
How often do we fixate?
What processing is done during fixation and during eye movement (saccade)?
Does the fixation duration indicate a learning concern?
   To answer such questions, linguistic theory is considered. Most of the text written in human languages has the following two main components: lexicon (a catalogue of language words); and grammar (a system or rules which allow for words to be combined into meaningful sentences).

Normally a word (term) can be in spoken in a language or written in a language. A word is made up of a prefix, a root word and a suffix. The root word may be an aggregation of one or more morphemes. A morpheme is a smallest meaningful grammatical unit in the English language. A morpheme is used to express a specific idea. The dependent morpheme in combination with other standalone morpheme refines the meaning of the standalone morpheme. Normally, non-native English readers, while reading novel word, tends to decode initially the meaning of morphemes and subsequently the meaning of the root word. Due to this reason, users tend to fixate on parts of a novel word multiple times, wherein each fixation could be of a longer duration. Therefore, the decoding of such novel words may require multiple fixations. Also, readers with reading disorders (such as dyslexia) read at a syllable level rather than at a word level. The reader's reading patterns result in an increased number of longer fixations as compared to reading patterns of readers without any reading impairments. The total fixation duration on a specific word is called as a gaze duration. A longer gaze duration indicates an interest in the term or concept. A longer gaze duration may be an indicator of a learning concern.

Subjective Word Familiarity And Word Frequency Influences Reading Behavior

All words do not tend to attract fixation. When the learner is familiar with the big word (word length>8 characters), the learner's eyes tend to have fewer, shorter fixations on the big word in comparison to novel or low familiar words of the same length. Few such high frequency words in a text may not attract any fixations because such terms may be treated as sight words or familiar words. Sight words or familiar words comprise high frequency words that are memorized as a whole such that they can be recognized without the need for decoding. Sight words, high frequency words and/or high familiar words do not always attract fixations.

Normally, the processing of words having higher frequency is quicker than lower frequency words. The same behavior was observed for familiar words. Words are classified as high frequency words or low frequency words based on objective frequency counts derived from text-based corpuses. Researchers have been using various word corpuses to classify words based on their frequency in online documents. Word corpuses include, but are not limited to, Corpus of Contemporary American English (“COCA”), a NOW corpus, and Wikipedia. Printed estimates of word frequency can be used as a word familiarity measure in order to classify novel words, low frequency words, and/or high frequency words. This means that low frequency words are considered as being less familiar than high frequency words. Word familiarity measures which are widely used for classifying words as familiar or unfamiliar include (1) printed estimates of word frequency and (2) subjective ratings of familiarity.

Term/Concept Classes

The present solution uses printed estimates of word frequency to derive word familiarity. Since word familiarity influences the number and duration of fixations, saccades, regression, pupillary diameter and recurrence, it is of upmost importance to know the familiarity status of a target word in order to predict a learning concern. Unlike prior research, the present solution first classifies words based on word frequency count plus word length. The words can be categorized based on a plurality of word frequency categories and a plurality of word length categories. The word frequency categories include, but are not limited to, a high frequency word category, a low frequency word category, a common word category, and a novel word category. The word length categories include, but are not limited to, a big-size word category, a mid-size word category, and a small-size word category. A word having a word length greater than eight characters is considered a big-size word. A word having a word length greater than three characters and greater than or equal to seven characters is considered a mid-size word. A word having a word length less than or equal to three characters is considered a small-size word. The combination of these categories results in the following twelve word categories: big-size/high-frequency; big-size/low-frequency; big-size/common-word; big-size/novel-word; mid-size/high-frequency; mid-size/low-frequency; mid-size/common-word; mid-size/novel-word; small-size/high-frequency; small-size/low-frequency; small-size/common-word; and small-size/novel-word.

Eye Movement and Pupillary Response Analysis

During a learner's reading survey, the learner's reading parameter data is collected (e.g., eye response data). Personalized reading threshold values for each word category are computed based on the collected learner's reading parameter data. Apart from using personalized threshold values of various biometric parameters (e.g., eye response signals) (local decision), the present solution also analyzes the effects of subjective word familiarity among a common class of learners (global decisions) in order to predict levels of learning.

During the prediction phase, while the learner is taking a course, the learner's reading behavior is recorded during lexical assess and syntactic processing of sentences (term-response map and concept-response map). Eye response signals collected during an initial processing of the target word and a reanalysis of the target word are considered tools for analyzing reading behavior of the learner during lexical assess and text comprehension. Eye response metrics (such as single fixation duration, first fixation duration, gaze duration, mean fixation duration, saccade length and spill over) are used to measure an initial processing time spent on a target word/concept. A second/subsequent pass time and a number of regressions are used to measure reanalysis. All of these following metrics are collectively analyzed to predict a learning concern (predict novel term/concept).
Single Fixation Duration (“SFD”): an amount of time spent when a reader makes only one fixation on a target word during an initial processing of the word.
First Fixation Duration (“FFD”): an amount of time spent by reader on a first fixation during an initial processing of the target word. In this case, a total number of fixations on a term/concept is greater than one.
Gaze Duration (“GD”): a sum of all consecutive fixation duration on a target word from a first fixation until a first time that a reader leaves the word.
Mean Fixation Duration (“MFD”): a mean of the sum of all fixation durations on a target word during an initial processing of the word.
Spill Over (“SO”): a duration of the fixation immediately following a reader's first pass fixations on a target word.
Second Pass Time (“SPT”): an amount of total processing time spent on the target word after exiting from the word and then returning to it later in time before navigating to a next slide.
Regressions: a number of look backs to a target word after a reader's initial encounter with the target word has ended.

Similarly, a pupillary diameter is also captured during fixations and saccades. The changes in pupillary diameter may be indicative of a higher cognitive load and a learning concern. Therefore, the following pupillary metrics are considered for prediction.
Mean pupil diameter of the left eye: a mean of all pupil diameters of a left eye that are recorded during the entire duration of a fixation on a specific term.
Mean Pupil diameter of the right eye: a mean of all pupil diameters of a right eye that are recorded during the entire duration of a fixation on a specific term

Contextual Information—Sensitivity Analysis

Based on the above basic model, the present solution is able to predict a novel term/concept or levels of learning, and provide assistive learning information. However, all novel terms do not require assistive learning information because readers normally process relevant contextual information (which may precede or follow the target word) in order to derive the meaning of a novel/low familiar word or concept. A reader's sensitivity to information context results in different reading patterns, wherein the reader exhibits more regressions out of the informative context during novel word processing in comparison to high frequency (familiar) word processing. On an occurrence of a high frequency/low frequency/novel word along with informative context, the reader may exhibit different reading patterns. Sometimes information context may be informative enough to help the reader derive meaning of novel or low familiar words. One argument is that these indicators do not necessarily mean that the informative context was really informative to infer the meaning of a novel word as readers normally engage in rereading the informative context during the processing of novel words. However, this ambiguity can be further ruled out by using novel-neutral context condition. In a novel-neutral condition, readers typically spend less initial processing time and less total time in the context region, and have fewer or no regressions in comparison to a related context condition. This shows that the readers did not spend more time in the neutral context since the neutral context did not add any new information in deriving the meaning of a novel word.

A reader's sensitivity to information context changes the eye response behavior. In order to increase a prediction accuracy of a learning concern, level of learning and/or AOI, the present solution analyzes a learner's information context processing behavior. Reading a novel or low familiar word may result in a similar reading pattern. One reason for this eye response behavior is that a lexical decision is normally a binary classification process. Mostly the word on initial encounter is considered as familiar or unfamiliar instead of being novel or low familiar and will receive similar attention on the first encounter. Moreover, it has been demonstrated that measures such as a total time spent and regressions in and out of a target word may be indicative to differentiate between novel and low familiar words. This indicates that on initial encounter, both novel and low frequency words seem to be unfamiliar. However, on further reexamination of informative text, the reader may be able to derive the meaning of the low familiar word by using past similar references from memory or past experiences. This may result in a larger number of regressions in and out of both the low frequency word and it corresponding informative context.

Recurrence Quantification Analysis

Re-examination of informative content or the target word may result in a recurrence of a fixation sequence or fixations on a term/concept. All re-fixations do not occur in a near future. Thus, the time of occurrence is highly important in this case. Therefore, to determine whether re-fixations occur close or far apart in the trial sequence, Recurrence Quantification Analysis (“RQA”) metrics (e.g., a Center of Recurrence Mass (“CORM”)) are used in order to predict whether an informative context/target word was re-examined closer or farther apart in a trial. Further during syntactic parsing of sentences (paragraph representing a concept), the learner's mental operations may detect and use cues in order to establish association between words. In this case, it is apparent that times of syntactic parsing will require certain terms/phrases of the sentences to be revisited in fine detail to comprehend its meaning whereas at occasions it may require a re-glance at those terms/phrases which were earlier read in fine detail to confirm the perceived meaning of the novel term/concept.

In order to measure these kind of fine temporal sequences of re-fixations as mentioned in these two cases, RQA metrics of laminarity are used. In another case, during syntactic parsing of a sentence, it may happen that sentences have associated words. Such lexical co-occurrence of novel words may trigger a recurrence of the sequence of fixations. Such recurrent fixations are detected by using determinism metrics. Recurrence and CORM metrics are used to capture the global temporal structure of fixation sequences. RQA metrics (such as Recurrence, Determinism, laminarity (lookback) and CORM) are used along with the above mentioned eye response metrics in order to increase prediction accuracy.

During text reading, readers do not always read every word as some readers are imaginers and some are verbalizers. The verbalizers read most of the words in a paragraph and have a less preview benefit. The imaginers have a large preview benefit and lower fixations.

Anticipatory Behavior Analysis

At times, while reading a part of a sentence or listening to the part of the sentence, the reader anticipates the upcoming input. It means readers predict upcoming input and react to it immediately even before receiving the bottom up processing information. The anticipatory reading behavior of learners often leads to varying reading patterns depending on whether the anticipated concept is similar to the actual concept or not. Hence, the reader's anticipatory behavior is analyzed by considering the regressions trigged due to anticipation outcomes.

Illustrative System Architecture

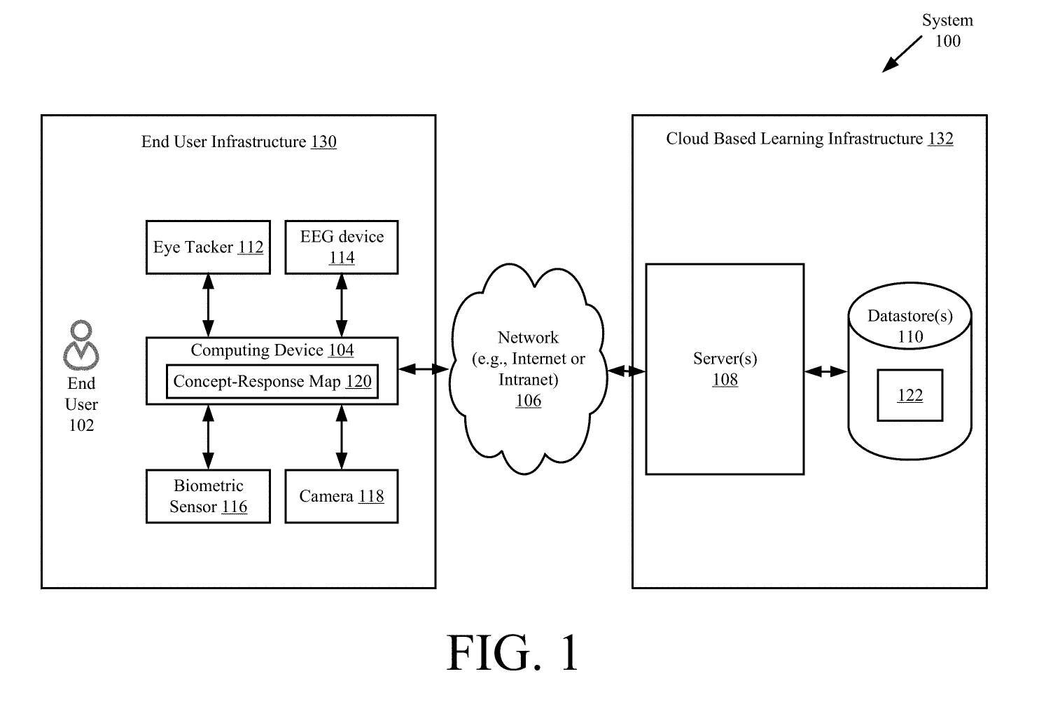

Referring now to FIG. 1, there is provided an illustrative system 100. System 100 is generally configured to provide a personalized learning experience to a user 102. The personalized learning experience is at least partially achieved using adaptive supplementary learning content. In this regard, system 100 performs an eye response analysis, a pupillary response analysis, a recurrent quantification analysis, an anticipatory behavior analysis, a contextual information sensitivity analysis, and an analysis of subjective word familiarity and word frequency in order to predict levels of learning. The supplementary learning content is then dynamically selected based on the predicted levels of learning and/or predicted AOCs or AOIs.

As shown in FIG. 1, system 100 comprises an end user infrastructure 130 and a cloud based learning infrastructure 132. The end user infrastructure 130 includes a computing device 104 facilitating cloud based learning by an end user 102 and a plurality of learning level indicator devices 112-118. The learning level indicator devices generally comprise HCI devices that track the cognitive, psychomotor and affective learning behavior of the user 102. The term “cognitive” means relating to cognition or the mental action or process of acquiring knowledge and understanding through thought, experience and the senses. The term “psychomotor” means relating to the origination of movement in conscious mental activity. The learning level indicator devices include, but are not limited to, an eye tracker 112, an EEG device 114, a biometric sensor 116, a camera 118, and/or a speaker (not shown). Each of the listed learning level indicator devices is well known in the art, and therefore will not be described herein. Any known or to be known eye tracker, EEG device, biometric sensor and/or a camera can be used herein without limitation.

During operation, the learning level indicator devices 112-118 generate observed sense data while the user 102 is taking several electronic reading surveys presented thereto via the computing device 104. The electronic reading surveys include content of different difficulty levels ranging from (i) text content having only common and high frequency words (terms), (ii) text content having combination of high and low frequency words (terms), (iii) text content having high, low frequency and novel words (terms), and/or (iv) multi-media content along with textual content. Notably, in some scenarios, the electronic reading surveys may be used in validating the method. In other scenarios, the user may be asked to read training text or the system may dynamically create a base line response.

Timestamped observed sense data is provided to computing device 104. The learning level indicator devices 112-118 can additionally or alternatively provide the timestamped observed sense data to the remote server 108 via network 106. The observed sense data can include, but is not limited to, eye response data, pupillary response data, neural signal data, facial expression data, heart rate data, temperature data, blood pressure data, and/or body part movement data (e.g., hand or arm movement). The observed sense data is analyzed by the computing device 104 and/or server 108 to predict a level of learning and/or at least one Area Of Concern (“AOC”) faced by the user 102 while reading. The AOC can include, but is not limited to, big-size/high-frequency words, big-size/low-frequency words, big-size/common-words, big-size/novel-words, mid-size/high-frequency words, mid-size/low-frequency words, mid-size/common-words, mid-size/novel-words, small-size/high-frequency words, small-size/low-frequency words, small-size/common-words, small-size/novel-words, high familiar concepts, novel concepts, and low familiar concepts. The learning assessment of all users of the cloud based learning system 100 is analyzed by the server 108 to collectively classify the users in different groups based on their levels of learning.

The AOC prediction is achieved using term/concept-response maps derived for observed behavior patterns of the user and a machine learning model. The machine learning model is trained with known behavior patterns of the user defined by training sense data. The training sense data is acquired while the user 102 performs at least one test survey. The training sense data is analyzed to determine a plurality of threshold values for each of a plurality of word (or term) categories and each of a plurality of concept categories. The word (or term) categories include, but are not limited to, (1) a big-size/high-frequency word category, (2) a big-size/low-frequency word category, (3) a big-size/common word category, (4) a big-size/novel word category, (5) a mid-size/high-frequency word category, (6) a mid-size/low-frequency word category, (7) a mid-size/common word category, (8) a mid-size/novel work category, (9) a small-size/high-frequency word category, (10) a small-size/low-frequency word category, (11) a small-size/common word category, and/or (12) a small-size/novel word category. The concept categories include, but are not limited to, a high familiar concept category, a low familiar concept category, and a novel concept category.

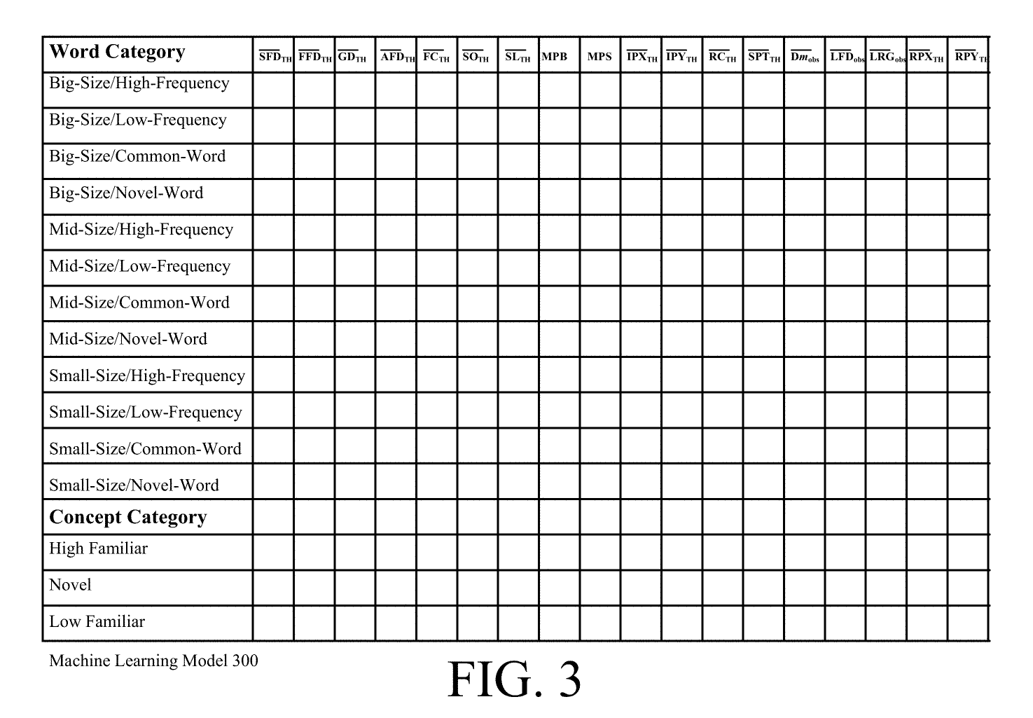

For example, first eye response signals are generated while the user takes a first look at the test survey, and second eye response signals are generated while the user takes a second subsequent look at the test survey. The first eye response signals are analyzed to determine the following items for each of a plurality of word (or term) categories and each of a plurality of concept categories: a mean Fixation Duration (“FD”) threshold S̅F̅D̅_{Th}; a mean first FD threshold F̅F̅D̅_{Th}; a gaze duration threshold G̅D̅_{Th}; an average FD threshold A̅F̅D̅_{Th}; a mean fixation count threshold F̅C̅_{Th}; a mean spillover threshold S̅O̅_{Th}; a mean Saccade Length (“SL”) threshold S̅L̅_{Th}; a Mean Preview Benefit (“MPB”); a Mean Perceptual Span (“MPS”); a mean pupil diameter of the left eye threshold I̅P̅X̅_{Th}; and a mean pupil diameter of the right eye threshold I̅P̅Y̅_{Th}. The second eye responses are analyzed to determine the following items for each of the word (or term) categories and each the concept categories: a mean regression count R̅C̅_{Th}; a mean second pass time S̅P̅T̅_{Th}; a determinism observed D̅m̅_{obs}; lookback fine detail observed L̅F̅D̅_{obs}; a lookback re-glance observed L̅R̅G̅_{obs}; a mean reanalysis pupil diameter of the left eye threshold R̅P̅X̅_{Th}; and a mean reanalysis pupil diameter of the right eye threshold R̅P̅Y̅_{Th}. Techniques for determining or computing each of these listed metric values are well known in the art, and therefore will not be described herein. Any known technique for determining and/or computing a mean single FD value, a mean fixation count value, a mean gaze duration value, a mean average FD value, a mean fixation count value, a mean spillover value, a mean SL value, an MPB value, an MPS value, a mean pupil diameter of the left eye value, a mean pupil diameter of the right eye value, a mean regression count value, a mean second pass time value, a determinism observed value, a lookback fine detail observed value, a lookback re-glance observed value, a mean reanalysis pupil diameter of the left eye value, and/or a mean reanalysis pupil diameter of the right eye value can be used herein without limitation. The table shown in FIG. 3 is useful for understanding an illustrative machine learning model 300. The present solution is not limited to the particulars of this example and the contents of FIG. 3. The machine learning model is used as baseline results in order to predict a learning difficulty and various levels of learning during an actual learning experience.

The term “fixation”, as used herein, means that both eyes of a person are fixated steadily on a point of interest (e.g., to read content). During fixation, the fovea of both eyes is steadily placed on the same location momentarily to read the content from that location. The term “fixation duration” or “FD”, as used herein, means the time duration for which a person steadily fixates at a fixation point. The FD can be measured in milliseconds. The term “saccade”, as used herein, refers to a rapid movement of an eye between fixed points. The term “saccade length” or “SL”, as used herein, refers to a distance between two consecutive fixations or the distance between two fixed points between which an eye rapidly moves. The SL is measured in characters in the case of a text content analysis. The term “preview benefit”, as used herein, refers to a total number of letters and/or words found between two subsequent fixation points. The term “perceptual span”, as used herein, refers to the total number of letters read from a left side to a right side of a fixation point. The perceptual span is dependent on the writing system used in the reading content. The term “regression”, as used herein, means the re-reading of text from a few words/sentences backwards in content.

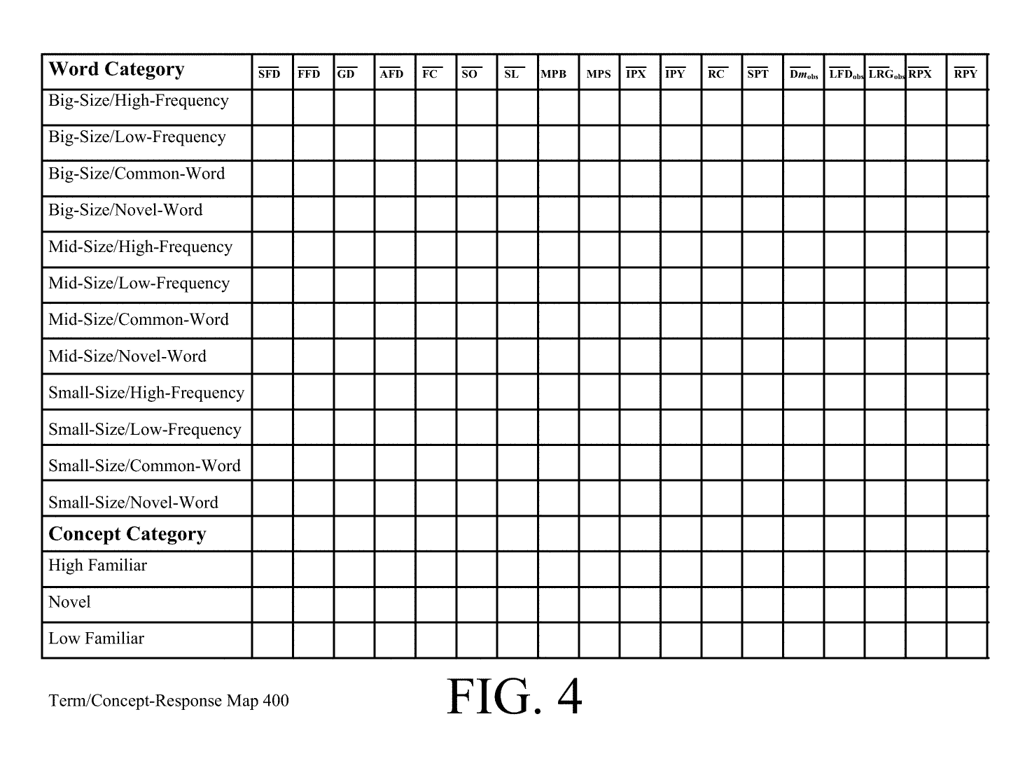

During the actual learning experience, observed sense data is acquired while the user performs at least one electronic reading survey presented thereto via the computing device 104. The observed sense data is analyzed to generate at least one term/concept-response map. The term/concept-response map is similar to the table shown in FIG. 3 but comprises values derived from the observed sense data rather than the training sense data. An illustration of an illustrative term/concept-response map 400 is shown in FIG. 4. The term/concept-response map is compared to the baseline results of the machine learning model. The results of this comparison are used to predict various levels of learning and/or at least one AOI/AOC.

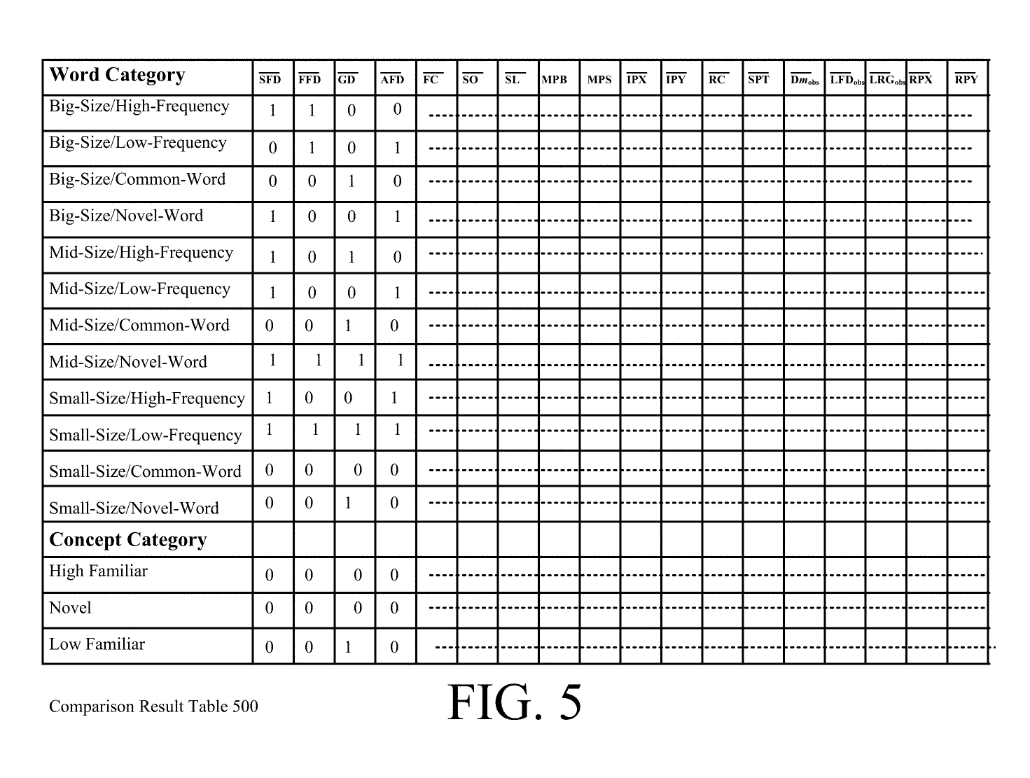

For example, the comparison involves determining if each of the values in the term/concept response map are greater than or equal to the respective threshold value contained in the machine learning model. If a value is greater than or equal to the respective threshold value, then a “1” is assigned to the corresponding biometric metric. Otherwise, a “0” is assigned thereto. A comparison result table 500 is generated that includes the 1's and 0's. An illustration of an illustrative comparison result table 500 is provided in FIG. 5. Next, the contents of the comparison result table is used to detect word categories and/or concept categories that are of concern. The following Mathematical Equation (1) defines an illustrative process for detecting a word or concept category of concern.

Cₓ=M₁+w₂·M₂+ . . . +w_{N}·M_{N} (1)

where Cₓ represents a result value associated with a given word or concept category, M₁-M_{N} each represent a binary value for a given metric, and w₁-w_{N} represents weights. The weights w₁-w_{N} are pre-defined fixed values derived for a given individual or a given group of individuals. An AOC is detected when the value of C₁ exceeds a given threshold value thr. The present solution is not limited to the particulars of this example. Other techniques can be employed to detect an AOC.

Predicting Learning Concerns Based on Learner's Eye Response Data

Human vision is divided into the following three regions: (i) foveal; (ii) parafoveal; and (iii) peripheral vision. Acuity of vision is the highest in the foveal region and gradually decreases from foveal to peripheral region. An AOI always attracts higher acuity. Therefore, eye movements known as saccades are performed to place fovea on the AOI. When the fovea is fixed at a point in the AOI, the point is normally termed as fixation. During a fixation, new information is normally read and not during saccades. So whenever a learner experiences difficulty to comprehend any term/concept, it may result in more fixations of longer duration and shorter saccades. A mean FD for skilled reader ranges from 225 ms to 250 ms during silent reading, whereas it ranges from 275 ms to 325 ms in the case of oral reading. The FD varies, and this could range from 50-75 ms to 500-600 ms in some scenarios. Shorter FDs can be due to reasons such as skipped reading, occurrence of sight words, and the reader's greater familiarity with the text (which requires less decoding time). In this case, the subsequent saccade lengths may eventually be longer. In contrast, longer FDs could be a result of encountering difficult text or group of words, which may require a longer time for decoding the word. In view of the forgoing, FD is used as one of the learning difficultly indicators or indicators of learning concern.

There is one exception. Cognitive processing of previously acquired information may continue during a saccade. This is the time taken for moving the eyes from a present fixation point Xi to a subsequent point Xi+1. Even though the FD at point Xi was shorter below the threshold, the information processing may have been carried out during the subsequent saccade or during the next fixation point Xi+1. Thus, the fixation point Xi, in spite of having shorter fixation, may be an AOI. In order to clearly identify the AOI, the SL and FD are considered learning difficulty indicators.

A mean SL of skilled English reader was found to be 2 degrees (i.e., 7-9 letters) during silent reading and 1.5 degrees (i.e., 6-7 letters) during oral reading. But at the same times, the SL can also vary from 1 letter space to 10-15 letter spaces. Accordingly, longer saccade length may be due to the reader's familiarity with the text, which is found between two subsequent fixation points or due to reader's familiarity with the text falling within the region of preview benefit. Hence, whenever the FD at a current fixation point Xi is longer than the threshold and the FD is shorter at the previous fixation point Xi−1 then the threshold, then two levels of learning may exist. First, if the FD at the previous fixation point Xi−1 is shorter and the subsequent SL is also shorter, than both points Xi and Xi−1 may be AOIs. Second, if the FD is shorter at point Xi−1 but the subsequent SL is longer, than the point Xi may be an AOI. Therefore, a longer FD at the current fixation point can be an indicator of learning difficulty experienced by the reader. However, shorter fixations cannot be outright removed. So this ambiguity can be further removed by considering the SL between current fixation point Xi and previous fixation point Xi−1 as an indicator of a learning concern.

Regressions are considered as a third learning difficulty indicator or indicator of learning concern. Backward saccades occur when text is found difficult. The backward SL can vary from one word to a few words. For both short and long range regressions, the forward reading continues from the Initiating Point of the last Regression (“IPR”). The IPR may also be an AOI. However, the challenge to using the IPR as an AOI is the ability to distinguish return sweeps from the regressions. A return sweep occurs whenever a reader almost reaches the end of one line and moves the eyes to first word of the next line. Modern eye tracking devices may provide better accuracy in distinguishing reverse sweeps from regressions.

With regard to fixations, it has been found that word length and probability of fixating on a word has some correlation. This finding shows that words having lengths greater than 8 letters are mostly fixated and longer complex words are often refixated, whereas smaller words of size 2-3 letters are rarely fixated. So during reading, shorter words are generally skipped, longer words yield multiple fixations, and regular words have few-fixations only.

To summarize the above discussion, the present solution considers fixations, saccades, regressions and pupil diameters as potential learning difficulty indicators or indicators of a learning concern. Therefore, during every trial, the present solution collects the learner's following eye response during initial processing of every term/concept and also during reanalysis of the term/concept.

The following eye response signals are recorded during the initial processing of the i^{th} term concept: a single fixation duration S̅F̅D̅ᵢ; a first fixation duration F̅F̅D̅ᵢ; a gaze duration G̅D̅ᵢ; a mean fixation duration A̅D̅F̅ᵢ; a fixation count F̅C̅ᵢ; a spillover S̅O̅ᵢ; a mean SL S̅L̅ᵢ; a preview benefit; and a perceptual span. The following pupil diameter values are recorded and computed during the initial processing of the i^{th} term concept: a mean pupil diameter of the left eye I̅P̅X̅ᵢ; and a mean pupil diameter of the right eye I̅P̅T̅ᵢ.

The following eye response signals are recorded during the reanalysis of the i^{th} term concept: a regression count R̅C̅ᵢ; a second pass time S̅P̅T̅ᵢ; a determinism observed D̅m̅ᵢ; a lookback fine detail observed L̅F̅D̅ᵢ; and a lookback re-glance observed L̅R̅G̅ᵢ. The following pupil diameter values are recorded and computed during the reanalysis of the i^{th} term concept: a mean pupil diameter of the left eye R̅P̅X̅ᵢ; and a mean pupil diameter of the right eye R̅P̅Y̅ᵢ.

Thereafter, the i^{th} term is classified into one of the 12 classes (e.g., big-size/high frequency, big-size/low frequency, big-size/common-word, big-size/common-novel, mid-size/high-frequency, mid-size/low frequency, mid-size/common-word, mid-size/common-novel, small-size/high frequency, small-size/low frequency, small-size/common-word, or small-size/common-novel) and/or the i^{th} concept is classified into one of 3 classes (high familiar, novel, or low familiar).

The resulting term/concept-response map is compared with the related baseline machine learning model. The levels of learning prediction process checks whether the above mentioned eye response values are greater than their corresponding threshold values. If so, then the respective indicator is set to true. For example, the i^{th} term belongs to the class big-size/low-frequency and has only one fixation. In this case, the i^{th} term's single fixation duration is greater than the related SFD threshold. Accordingly, the SFD outcome variable is set to 1. The logic is defined by the following Mathematical Equation (2).

If S̅F̅D̅ᵢ>S̅F̅D̅ᵢ(big-size/low-frequency)→S̅F̅D̅ᵢ(out)=1 (2)

The example shows that the i^{th} term belongs to the big-size/low-frequency class, and that the single fixation duration-outcome variable is set to one if the term has attracted a single fixation that is greater than the corresponding threshold value of the machine learning model. This means that the SFD metric indicates a learning concern. The same process is carried out for all metrics. Thereafter, a majority voting method is used to do binary classification of the term/concept into a Learning Concern Detected (“LCD”) class or a No Learning Concern Detected (“NLCD”) class. Finally, the term/concept-response map related to the predicted learning concern will update the learner's machine learning model. Hence, the machine learning model is updated after new discovery of a reading behavior which may contribute to an increase in the prediction accuracy for later trials.

Based on the predicted level of learning, the related e-content for the learner is dynamically modified. Related Assistive Supplementary e-learning content is then presented to the learner. A Global Learning Assessment (“GLA”) of a plurality of learners is also performed. The GLA classifies learners into various learner groups based on their levels of learning. The term/concept-response maps are analyzed in order to classify learners in various groups. Classification algorithms (e.g., naïve Bayes) may be used in order to increase classification accuracy. Accordingly, the present solution uses local and global adaptive behavior to assist the learner with supplementary adaptive learning content in real time.

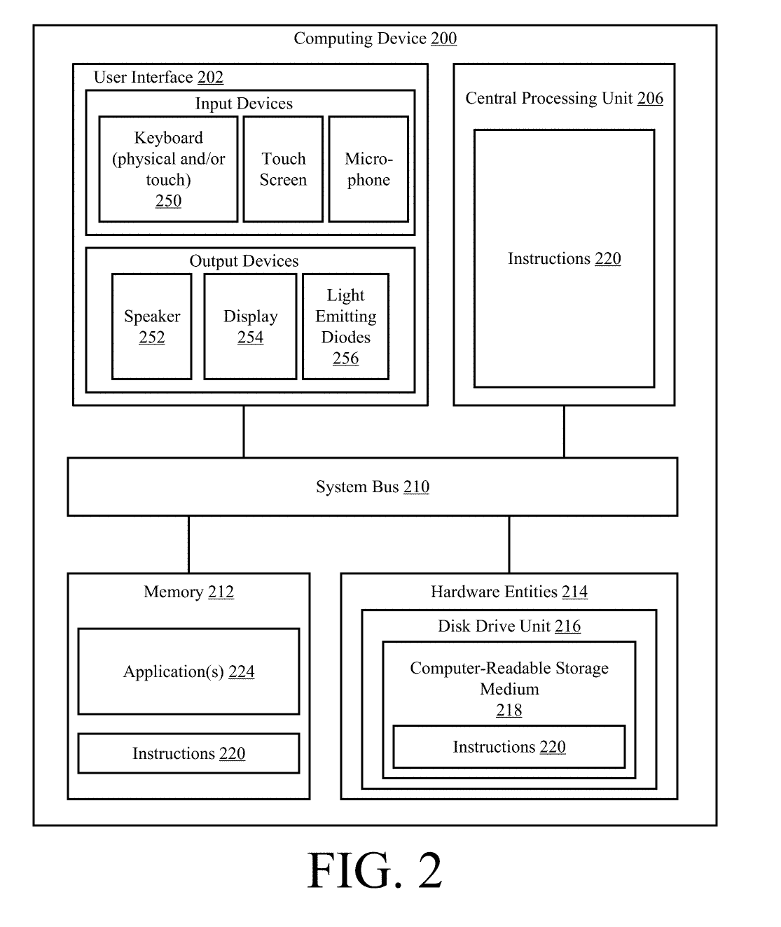

Referring now to FIG. 2, there is provided an illustration of an exemplary architecture for a computing device 200. Computing device 104 and/or server(s) 108 of FIG. 1 (is) are the same as or similar to computing device 200. As such, the discussion of computing device 200 is sufficient for understanding these components of system 100.

Computing device 200 may include more or less components than those shown in FIG. 2. However, the components shown are sufficient to disclose an illustrative solution implementing the present solution. The hardware architecture of FIG. 2 represents one implementation of a representative computing device configured to enable watermarking of graphics, as described herein. As such, the computing device 200 of FIG. 2 implements at least a portion of the method(s) described herein.

Some or all the components of the computing device 200 can be implemented as hardware, software and/or a combination of hardware and software. The hardware includes, but is not limited to, one or more electronic circuits. The electronic circuits can include, but are not limited to, passive components (e.g., resistors and capacitors) and/or active components (e.g., amplifiers and/or microprocessors). The passive and/or active components can be adapted to, arranged to and/or programmed to perform one or more of the methodologies, procedures, or functions described herein.

As shown in FIG. 2, the computing device 200 comprises a user interface 202, a Central Processing Unit (“CPU”) 206, a system bus 210, a memory 212 connected to and accessible by other portions of computing device 200 through system bus 210, and hardware entities 214 connected to system bus 210. The user interface can include input devices and output devices, which facilitate user-software interactions for controlling operations of the computing device 200. The input devices include, but are not limited, a physical and/or touch keyboard 250. The input devices can be connected to the computing device 200 via a wired or wireless connection (e.g., a Bluetooth® connection). The output devices include, but are not limited to, a speaker 252, a display 254, and/or light emitting diodes 256.

At least some of the hardware entities 214 perform actions involving access to and use of memory 212, which can be a Random Access Memory (“RAM”), a disk driver and/or a Compact Disc Read Only Memory (“CD-ROM”). Hardware entities 214 can include a disk drive unit 216 comprising a computer-readable storage medium 218 on which is stored one or more sets of instructions 220 (e.g., software code) configured to implement one or more of the methodologies, procedures, or functions described herein. The instructions 220 can also reside, completely or at least partially, within the memory 212 and/or within the CPU 206 during execution thereof by the computing device 200. The memory 212 and the CPU 206 also can constitute machine-readable media. The term “machine-readable media”, as used here, refers to a single medium or multiple media (e.g., a centralized or distributed database, and/or associated caches and servers) that store the one or more sets of instructions 220. The term “machine-readable media”, as used here, also refers to any medium that is capable of storing, encoding or carrying a set of instructions 220 for execution by the computing device 200 and that cause the computing device 200 to perform any one or more of the methodologies of the present disclosure.

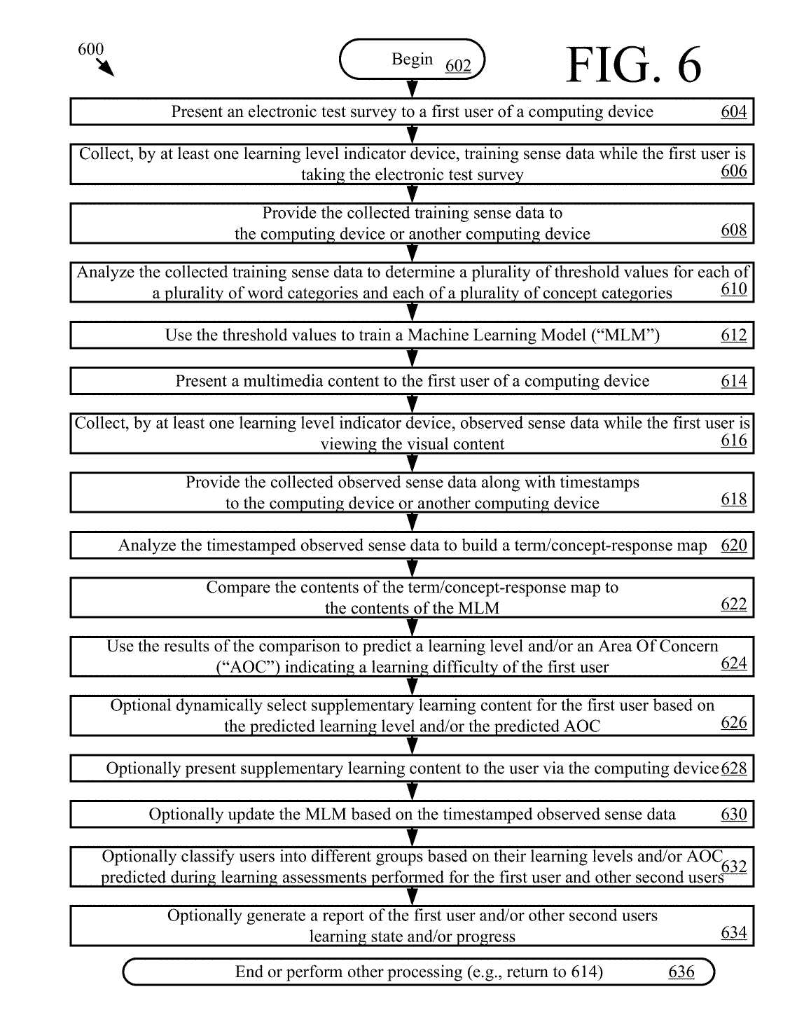

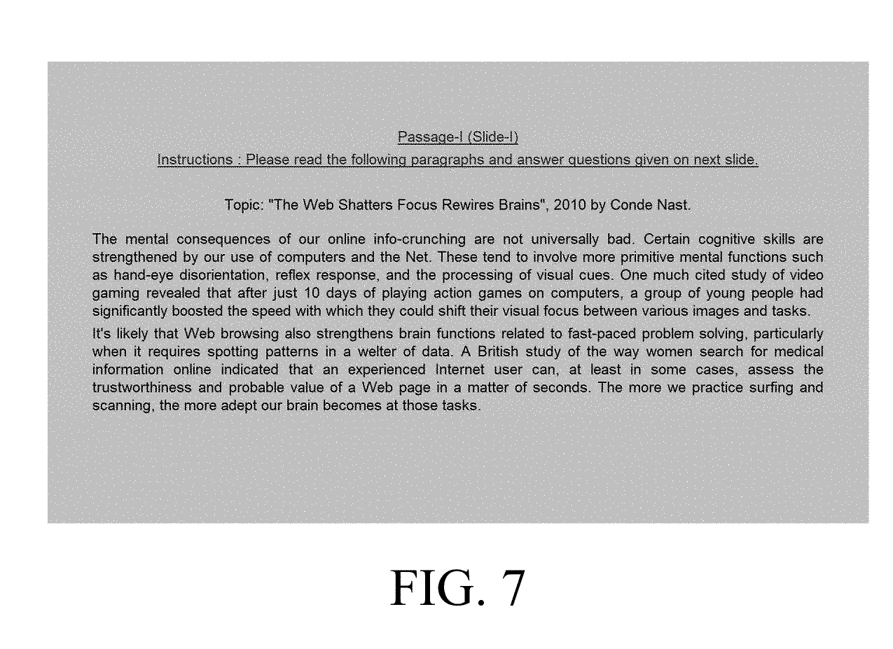

Referring now to FIG. 6, there is provided a flow diagram of an illustrative method 600 for predicting a person's learning level and/or reading disorder. Method 600 begins with 602 and continues with 604 where an electronic test survey is presented to a first user (e.g., user 102 of FIG. 1) of a computing device (e.g., computing device 104 of FIG. 1). The computing device can include, but is not limited to, a desktop computer, a laptop computer, a smart device (e.g., a smart phone), a wearable computing device (e.g., a smart watch), and/or a personal digital assistant. The electronic test survey is presented via a display screen (e.g., display screen 254 of FIG. 2) of the computing device. An illustration of an illustrative electronic test survey 700 is provided in FIG. 7.

As shown by 606, at least one learning level indicator device (e.g., device(s) 112, 114, 116 and/or 118 of FIG. 1) collects training sense data while the first user is taking the electronic test survey. The collected training sense data is provided to the computing device or another computing device (e.g., server 108 of FIG. 1) in 608. The collected training sense data is analyzed in 610 to determine a plurality of threshold values for each of a plurality of word categories and each of a plurality of concept categories. The word categories include, but are not limited to, a big-size/high-frequency word category, a big-size/low-frequency word category, a big-size/common-word category, a big-size/novel-word category, a mid-size/high-frequency word category, a mid-size/low-frequency word category, a mid-size/common-word category, a mid-size/novel-word category, a small-size/high-frequency word category, a small-size/low-frequency word category, a small-size/common-word category, and/or a small-size/novel-word category. The concept categories include, but are not limited to, a high familiar category, a novel category, and a low familiar category. The threshold values are used in 612 to train a machine learning model (e.g., machine learning model 300 of FIG. 3). In some scenarios, 612 involves populating a table with determined and/or computed metric threshold values. The metric threshold values include, but are not limited to, a mean single FD threshold value S̅F̅D̅_{Th}, a mean first FD threshold value F̅F̅D̅_{Th}, a mean gaze duration threshold value G̅D̅_{Th}, a mean average FD threshold value A̅F̅D̅_{Th}, a mean fixation count threshold value F̅C̅_{Th}, a mean spillover threshold value S̅O̅_{Th}, a mean SL threshold value S̅L̅_{Th}, an MPB value, an MPS value, a mean pupil diameter of the left eye threshold value I̅P̅X̅_{Th}, a mean pupil diameter of the right eye threshold value I̅P̅Y̅_{Th}, a mean regression count value R̅C̅_{Th}, a mean second pass time value S̅P̅T̅_{Th}, a determinism observed value D̅m̅_{obs}, a lookback fine detail observed value L̅F̅D̅_{obs}, a lookback re-glance observed value L̅R̅G̅_{obs}, a mean reanalysis pupil diameter of the left eye threshold value R̅P̅X̅_{Th}, and a mean reanalysis pupil diameter of the right eye threshold value R̅P̅Y̅_{Th}. The present solution is not limited to the particulars of these scenarios.

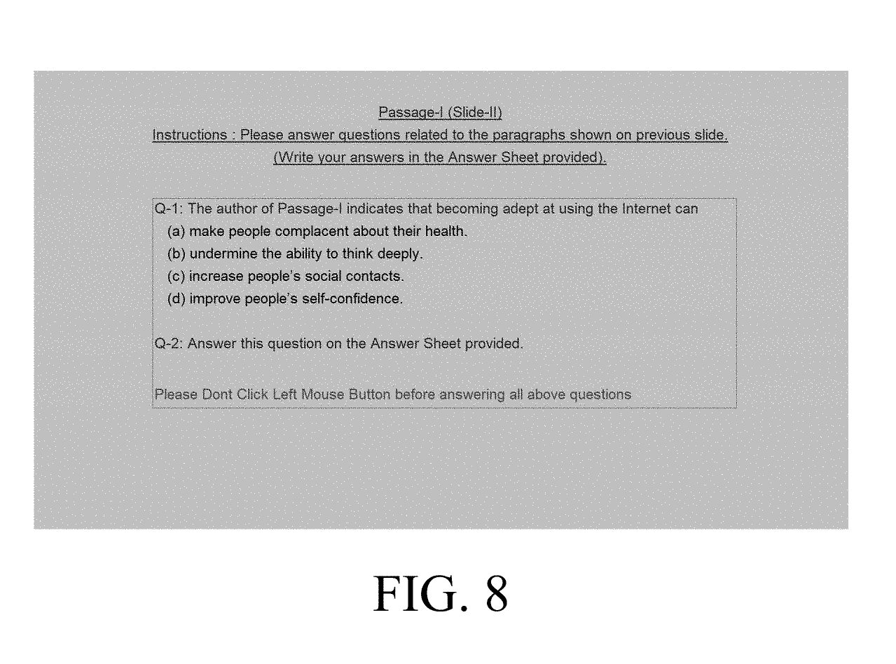

Thereafter, operations are performed to assess the first user's learning ability. In this regard, method 600 continues with 614 where multimedia content is presented to the first user. The multimedia content is presented via a display screen (e.g., display screen 254 of FIG. 2) of the computing device (e.g., computing device 104 of FIG. 1). An illustration of an illustrative displayed multimedia content is provided in FIG. 8.

As shown by 616, at least one learning level indicator device (e.g., device(s) 112, 114, 116 and/or 118 of FIG. 1) collects observed sense data while the first user is viewing the visual content. The collected observed sense data is provided to the computing device or another computing device (e.g., server 108 of FIG. 1) in 618. The collected observed sense data is analyzed in 620 to build a term/concept-response map (e.g., term/concept-response map 400 of FIG. 4). The term/concept-response map is built by determining a plurality of metric values for each of a plurality of word categories and each of a plurality of concept categories. The metric values include, but are not limited to, a single fixation duration value S̅F̅D̅ᵢ, a first fixation duration value F̅F̅D̅ᵢ, a gaze duration value G̅D̅ᵢ, a mean fixation duration value A̅F̅D̅ᵢ, a fixation count value F̅C̅ᵢ, a spillover value S̅O̅ᵢ, a mean SL value S̅L̅ᵢ, a preview benefit value, a perceptual span value, a mean pupil diameter of the left eye value I̅P̅X̅ᵢ, a mean pupil diameter of the right eye value I̅P̅Y̅ᵢ, a regression count value R̅C̅ᵢ, a second pass time value S̅P̅T̅ᵢ, a determinism observed value D̅m̅ᵢ, a lookback fine detail observed value L̅F̅D̅ᵢ, a lookback re-glance observed value L̅R̅G̅ᵢ, a mean reanalysis pupil diameter of the left eye value R̅P̅X̅ᵢ, and a mean reanalysis pupil diameter of the right eye value R̅P̅Y̅ᵢ. The metric values can then be used to populate a table.

Next in 622, the content of the term/concept-response map is compared to the content of the machine learning model. In some scenarios, the comparison operation involves comparing each given metric value of the term/concept-response map to a respective metric threshold value contained in the machine learning model. The result of the comparison operations are used in 624 to predict a learning level and/or an AOC indicating a learning difficulty of the first user. In some scenarios, 624 involves: assigning a “1” value or a “0” value to each metric based on results of the comparison operations; populating a comparison result table (e.g., comparison result table 500 of FIG. 1) with the assigned “1” values and “0” values; computing a result value Cₓ for each word category and each concept category in accordance with Mathematical Equation (1) provided above; respectively comparing the result values to threshold values; and detecting an AOC when a result value is equal to or exceeds the respective threshold value. Upon completing 624, various actions can be taken.

In some scenarios, method 400 continues with optional blocks 626-628. These blocks involve: dynamically selecting supplementary learning content for the first user based on the predicted learning level and/or the predicted AOC; and present the dynamically selected supplementary learning content to the user via the computing device. The following operations may additionally or alternatively be performed: updating the machine learning model based on the timestamped observed sense data as shown by 630; classifying users into different groups based on their learning levels and/or AOC predicted during learning assessments performed for the first user and other second users as shown by 632; and/or generating a report of the first user and/or second users learning state and/or progress as shown by 634. Subsequently, 636 is performed where method 600 ends or other processing is performed.

Although the present solution has been illustrated and described with respect to one or more implementations, equivalent alterations and modifications will occur to others skilled in the art upon the reading and understanding of this specification and the annexed drawings. In addition, while a particular feature of the present solution may have been disclosed with respect to only one of several implementations, such feature may be combined with one or more other features of the other implementations as may be desired and advantageous for any given or particular application. Thus, the breadth and scope of the present solution should not be limited by any of the above described embodiments. Rather, the scope of the present solution should be defined in accordance with the following claims and their equivalents.

What is claimed is:

1. A method for predicting at least one of a user's learning level and Area Of Concern (“AOC”), comprising:
presenting multimedia content to a user of a computing device;
collecting, by at least one learning level indicator device, observed sense data specifying the user's behavior while the user views the multimedia content;
analyzing the observed sense data to determine a plurality of metric values for each of a plurality of word categories; and
using the metric values for predicting at least one of the learning level and the AOC.

2. The method according to claim 1, wherein the metric values are used in a previously trained machine learning model for predicting at least one of the learning level and the AOC.

3. The method according to claim 1, wherein a machine learning model is trained with observed sense data collected while a user is presented with training multimedia content.

4. The method according to claim 1, wherein a machine learning model is trained with observed sense data collected from a plurality of users while each user is presented with training multimedia content.

5. The method according to claim 1, wherein the at least one learning level indicator device comprises at least one of an eye tracker, an Electroencephalogram, a biometric sensor, a camera, and a speaker.

6. The method according to claim 1, wherein the plurality of metric values comprises at least one of a single fixation duration value, a first fixation duration value, a gaze duration value, a mean fixation duration value, a fixation count value, a spillover value, a mean saccade length value, a preview benefit value, a perceptual span value, a mean pupil diameter of a left eye value, a mean pupil diameter of a right eye value, a regression count value, a second pass time value, a determinism observed value, a lookback fine detail observed value, a lookback re-glance observed value, a mean reanalysis pupil diameter of the left eye value, and a mean reanalysis pupil diameter of the right eye value.

7. The method according to claim 1, wherein the plurality of word categories comprises a big-size/high-frequency word category, a big-size/low-frequency word category, a big-size/common-word category, a big-size/novel-word category, a mid-size/high-frequency word category, a mid-size/low-frequency word category, a mid-size/common-word category, a mid-size/novel-word category, a small-size/high-frequency word category, a small-size/low-frequency word category, a small-size/common-word category, and/or a small-size/novel-word category.

8. The method according to claim 1, wherein the metric values are also determined for a plurality of concept categories comprising a high familiar category, a novel category, and a low familiar category.

9. The method according to claim 1, further comprising dynamically selecting supplementary learning content for the user based on at least one of the predicted learning level and the predicted AOC.

10. The method according to claim 9, further comprising presenting the supplementary learning content to the user via the computing device.

11. The method according to claim 1, further comprising generating a report of at least one of the user's learning state and the user's progress based on at least one of the predicted learning level and the predicted AOC.

12. The method according to claim 3, wherein the training multimedia content comprises content of different difficulty levels ranging from (i) text content having only common and high frequency words, (ii) text content having combination of high and low frequency words, (iii) text content having high, low frequency and novel words, and (iv) multi-media content along with textual content.

13. A method for predicting at least one of a user's learning level and Area Of Concern (“AOC”), comprising:
presenting multimedia content to a user of a computing device;
collecting, by at least one learning level indicator device, observed sense data specifying the user's behavior while the user views the multimedia content;
analyzing the observed sense data to determine a plurality of metric values for each of a plurality of word categories; and
comparing the metric values obtained for the same word at different times for predicting at least one of the learning level and the AOC.

14. The method according to claim 13, wherein the metric values are used in a previously trained machine learning model for predicting at least one of the learning level and the AOC.

15. The method according to claim 13, wherein a machine learning model is trained with observed sense data collected while a user is presented with training multimedia content.

16. The method according to claim 13, wherein a machine learning model is trained with observed sense data collected from a plurality of users while each user is presented with training multimedia content.

17. A system, comprising:
a processor; and
a non-transitory computer-readable storage medium comprising programming instructions that are configured to cause the processor to implement a method for predicting at least one of a user's learning level and Area Of Concern (“AOC”), wherein the programming instructions comprise instructions to:
present multimedia content to a user of a computing device;
obtain observed sense data specifying the user's behavior which was collected by at least one learning level indicator device while the user views the multimedia content;
analyze the observed sense data to determine a plurality of metric values for each of a plurality of word categories and a plurality of concept categories;
compare the metric values respectively to metric threshold values of a machine learning model previously trained with training sense data specifying the user's behavior while taking an electronic test survey; and
predict at least one of the learning level and the AOC based on results of the comparing.

18. The system according to claim 17, wherein the at least one learning level indicator device comprises at least one of an eye tracker, an Electroencephalogram, a biometric sensor, a camera, and a speaker.

19. The system according to claim 17, wherein the plurality of metric values comprises at least one of a single fixation duration value, a first fixation duration value, a gaze duration value, a mean fixation duration value, a fixation count value, a spillover value, a mean saccade length value, a preview benefit value, a perceptual span value, a mean pupil diameter of a left eye value, a mean pupil diameter of a right eye value, a regression count value, a second pass time value, a determinism observed value, a lookback fine detail observed value, a lookback re-glance observed value, a mean reanalysis pupil diameter of the left eye value, and a mean reanalysis pupil diameter of the right eye value.

20. The system according to claim 17, wherein the plurality of word categories comprises a big-size/high-frequency word category, a big-size/low-frequency word category, a big-size/common-word category, a big-size/novel-word category, a mid-size/high-frequency word category, a mid-size/low-frequency word category, a mid-size/common-word category, a mid-size/novel-word category, a small-size/high-frequency word category, a small-size/low-frequency word category, a small-size/common-word category, and/or a small-size/novel-word category.

21. The system according to claim 17, wherein the plurality of concept categories comprises a high familiar category, a novel category, and a low familiar category.

22. The system according to claim 17, wherein the programming instructions further comprise instructions to dynamically select supplementary learning content for the user based on at least one of the predicted learning level and the predicted AOC.

23. The system according to claim 22, wherein the programming instructions further comprise instructions to present the supplementary learning content to the user.

24. The system according to claim 17, wherein the programming instructions further comprise instructions to update the machine learning model based on the observed sense data.

25. The system according to claim 17, wherein the programming instructions further comprise instructions to generate a report of at least one of the user's learning state and the user's progress based on at least one of the predicted learning level and the predicted AOC.

26. The system according to claim 17, wherein the electronic test survey comprises content of different difficulty levels ranging from (i) text content having only common and high frequency words, (ii) text content having combination of high and low frequency words, (iii) text content having high, low frequency and novel words, and (iv) multi-media content along with textual content.

27. A method for predicting at least one of a user's learning level and Area Of Concern (“AOC”), comprising:
presenting multimedia content to a user of a computing device;
collecting, by at least one learning level indicator device, observed sense data specifying the user's behavior while the user views the multimedia content;
analyzing the observed sense data to determine a plurality of metric values for each of a plurality of graphical element categories; and
using the metric values for predicting at least one of the learning level and the AOC.

28. A method for adapting content, comprising:
presenting multimedia content to a user of a computing device;
predicting, determining and calculating at least one of a level of learning and an area of concern; and
modifying the presented multimedia content based on at least one of the level of learning and the area of concern.

29. The method according to claim 28, wherein the multimedia content is modified by providing a supplementary content that clarifies the multimedia content.

30. The method according to claim 29, wherein the multimedia content is modified by providing definitions of one or more terms in the multimedia content.

31. A method for grouping learners, comprising:
presenting multimedia content to a user of a computing device;
predicting, determining and calculating at least one of a level of learning and an area of concern; and
creating a group of learners with at least one of a similar level of learning and a similar area of concern.

32. The method according to claim 31, wherein learners are grouped and placed in a common chat room.

33. The method according to claim 31, wherein learners are grouped and placed in a common online study space.
